(19) United States
(12) Patent Application Publication
(10) Pub. No.: US 2004/0230114 A1
(43) Pub. Date: Nov. 18, 2004

(54) MRI flex circuit catheter imaging coil

(76) Inventors: Guy V. Clatterbaugh, Columbia, MD (US); Paul A. Vichot, Ellicott City, MD (US); James G. Palmer, Ellicott City, MD (US); Henry R. Halperin, Baltimore, MD (US)

Correspondence Address: Office of Patent Counsel, The Johns Hopkins University, Applied Physics Laboratory, Mail Stop 7-156, 11100 Johns Hopkins Road, Laurel, MD, 20723-6099, US

(21) Appl. No.: 10/803,254

(22) Filed: Mar. 18, 2004

Related U.S. Application Data
(60) Provisional application No. 60/455,656, filed on Mar. 18, 2003.

(51) Int. Cl. A61B 6/00; A61M 25/00

(52) U.S. Cl. 600/435

(57) ABSTRACT

A method and structure for a radio frequency receiver coil adapted to be extended from a catheter. This coil comprises a flexible printed wiring board that has a first end and a second end extending from the opening in the catheter and a connection external to the catheter joining the first end to the second end to form a loop. The flexible printed wiring board also includes shielding circuitry (e.g., a Faraday shield) on the flexible printed wiring board. The first end is more flexible than the second end and the relative flexibility of the first end with respect to the second end causes the first end to take the shape of a round arc when extended from the catheter. In addition, insulator sections on the flexible printed wiring board define the shape of the loop. Independently moveable control rods are connected to the first end and the second end, to allow the first end to be extended further out of the opening than the second end (to form the loop).

### STATEMENT OF GOVERNMENTAL INTEREST

This invention was made with government support under grant number R01-HL64795-01 awarded by the National Institutes of Health under their U.S. Army contract number M400-217-2645. The government has certain rights in this invention.

### BACKGROUND OF THE INVENTION

1. Field of the Invention

The present invention comprises an imaging coil for use with radio frequency (RF) imaging devices and more particularly to a shielded coil positioned within a catheter used to pick up magnetic lines of flux during, for example, magnetic resonance imaging (MRI) procedures.

2. Description of the Related Art

Magnetic resonance technology provides safe, rapid images of a patient and produces chemical shift spectra to provide information regarding the chemical content of a material. In a general sense, magnetic resonance imaging involves providing bursts of radio frequency (RF) energy on a specimen positioned within a main magnetic field in order to induce responsive emission of magnetic radiation from the hydrogen nuclei or other nuclei.

The emitted signal may be detected in such a manner as to provide information as to the intensity of the response and the spatial original of the nuclei emitting the responsive magnetic resonance signal. In general, imaging may be performed in a slice or plane or multiple planes or three-dimensional volume with information corresponding to the responsively emitted magnetic radiation being received by a computer that stores the information in the form of numbers corresponding to the intensity of the signal. The pixel value may be established in the computer by employing Fourier Transformation that converts the signal amplitude as a function of time to signal amplitude as a function of frequency. The signals may be stored in the computer and may be delivered with or without enhancement to a video screen display, such as a cathode-ray tube, for example, wherein the image created by the computer output will be presented through black and white presentations varying in intensity or color presentations varying in hue and intensity. See, generally, U.S. Pat. No. 4,766,381.

Often, the responsive emission signal is detected using a coil comprising one or more loops of conductive wiring. The resolution of the image is increased by positioning the coil as close as possible to the item being damaged. Therefore, if the coil can be placed directly on or within an item, the MRI imaging resolution is dramatically increased. However, it is difficult to position a coil within certain items such as living organs or other small delicate items. For example, is difficult to position a coil within the heart of the living animal or human. Therefore, there is a need for a device (and method of using such a device) that allows a coil to be easily inserted and removed from small delicate items, without damaging the items, to allow increased resolution of MRI imaging. The invention described below addresses these needs.

### SUMMARY OF THE INVENTION

In one embodiment, the invention comprises a radio frequency receiver coil adapted to be extended from a catheter. This coil comprises a flexible printed wiring board that has a first end and a second end extending from the opening in the catheter and a connection external to the catheter joining the first end to the second end to form a loop. The flexible printed wiring board also includes shielding circuitry (e.g., a Faraday shield) on the flexible printed wiring board. The first end is more flexible than the second end and the relative flexibility of the first end with respect to the second end causes the first end to take the shape of a round arc when extended from the catheter. In addition, insulator sections on the flexible printed wiring board define the shape of the loop. Independently moveable control rods are connected to the first end and the second end to allow the first end to be extended further out of the opening than the second end (to form the loop). Also, the flexible printed wiring board includes capacitors adjacent the second end.

The invention provides a method of manufacturing this radio frequency receiver coil. This method first forms a flexible printed wiring board (that includes forming shielding circuitry on the flexible printed wiring board) and connects ends of the flexible printed wiring board together. The independently movable control rods are connected to the flexible printed wiring board next. Various wiring, spacer, etc. is then connected to the flexible printed wiring board to complete the coil. The flexible printed wiring board is then positioned within a catheter such that the ends of the flexible printed wiring board extend from the opening of the catheter. The control rods are moved to extend the first end of the flexible printed wiring board further out of the opening than the second end of the flexible printed wiring board such that the portion of the flexible printed wiring board outside the opening forms a loop.

The invention also provides a catheter that uses this radio frequency receiver coil. This catheter comprises an enclosed section having an opening and the radio frequency receiver coil is adapted to be extended from the opening of the catheter. Once again, the coil comprises a flexible printed wiring board having first and second ends, and a connection external to the catheter joining the first end to the second end to form a loop.

The structures and methods described herein provide a method of performing magnetic resonance imaging (MRI). In this methodology, a catheter is inserted into an item, such that an opening at one end of the catheter is positioned within the item. Next, the radio frequency coil comprising a flexible printed wiring board is inserted into the item through the catheter. A first control rod is moved to extend a first end of the flexible printed wiring board further out of the opening than a second end of the flexible printed wiring board, such that the portion of the flexible printed wiring board outside the opening forms a loop. Then, the invention senses a radio frequency signal generated outside the item using the radio frequency coil.

These, and other, aspects of the present invention will be better appreciated and understood when considered in conjunction with the following description and the accompanying drawings. It should be understood, however, that the following description, while indicating preferred embodiments of the present invention and numerous specific details thereof, is given by way of illustration and not of limitation. Many changes and modifications may be made within the scope of the present invention without departing from the spirit thereof, and the invention includes all such modifications.

### BRIEF DESCRIPTION OF THE DRAWINGS

The invention will be better understood from the following detailed description with reference to the drawings, in which:

FIG. 1 is a cross-sectional schematic diagram of the inventive catheter/coil in the undeployed configuration;

FIG. 2 is a cross-sectional schematic diagram of the inventive catheter/coil in the deployed configuration;

FIG. 3 is a top-view schematic diagram of the inventive coil illustrating the Faraday shield incorporated therein;

FIG. 4 is a top-view schematic diagram of the inventive coil illustrating the sections of insulator incorporated therein;

FIG. 5 is a schematic block diagram of a MRI system incorporating the invention;

FIG. 6 is a flow diagram illustrating a preferred method of the invention;

FIG. 7 is a flow diagram illustrating a preferred method of the invention;

FIG. 8 is a cross-sectional schematic diagram of one embodiment of the inventive coil; and.

FIG. 9 is a cross-sectional schematic diagram of one embodiment of the inventive coil.

### DETAILED DESCRIPTION OF PREFERRED EMBODIMENTS OF THE INVENTION

The present invention and the various features and advantageous details thereof are explained more fully with reference to the nonlimiting embodiments that are illustrated in the accompanying drawings and detailed in the following description. It should be noted that the features illustrated in the drawings are not necessarily drawn to scale. Descriptions of well-known components and processing techniques are omitted so as to not unnecessarily obscure the present invention. The examples used herein are intended merely to facilitate an understanding of ways in which the invention may be practiced and to further enable those of skill in the art to practice the invention. Accordingly, the examples should not be construed as limiting the scope of the invention.

The invention provides a catheter that uses a radio frequency receiver coil. This catheter comprises an enclosed section having an opening and a radio frequency receiver coil that extends from the opening of the catheter. The coil comprises a flexible printed wiring board having first and second ends, and a connection external to the catheter joining the first end to the second end to form a loop.

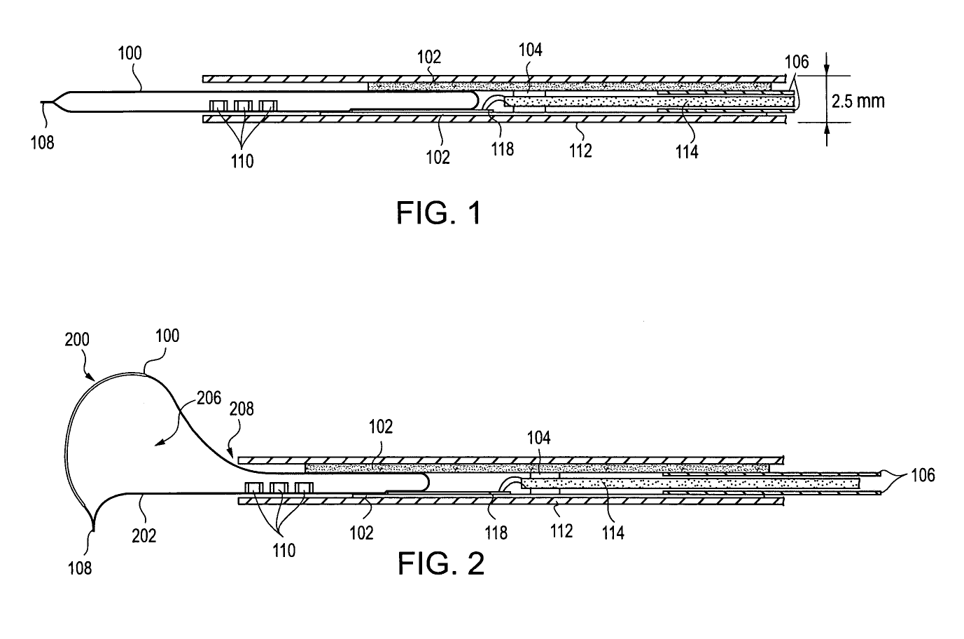

As shown in FIGS. 1 and 2, this structure includes a relatively long (e.g., 10-25 mm), narrow (e.g., 0.1-0.5 mm), thin (e.g., 0.01-0.1 mm) flexible piece of printed wiring board (PWB) 100 within a catheter 112 (e.g., an 8 French cardiac catheter). A radio frequency receiver coil 206 is formed by creating one or more loops 200, 202 of wiring. The loop 206 is designed to optimally receive the time dependent magnetic induction field stimulated by the RF pulse sequences generated during an MRI procedure for subsequent image processing. Thus, the length of the upper portion 200 of the printed wiring board with respect to the lower portion 202 is designed to provide an appropriately sized loop, again depending upon the specific MRI application at issue.

In the non-deployed position shown in FIG. 1, the printed wiring board 100 is narrower than the exterior of the catheter 112 and therefore can be positioned anywhere the catheter can be positioned (for example, within a blood vessel that opens into an organ, such as a heart). Once positioned inside the item to be imaged, the upper steering wire 106 and controlling rod 102 are moved toward the opening 208 of the catheter 112 to cause the upper portion of the printed wiring board 200 to extend from the catheter 112 and form the loop 206 shown in FIG. 2. After the imaging process is completed, the steering wire 106 and controlling rod 102 are retracted away from the opening 208 of the catheter 112 causing the upper portion of the printed wiring board 200 to also be retracted within the catheter 112 to the original position shown in FIG. 1. This allows the inventive catheter/coil structure to be inserted within and removed from an item being imaged without damaging the item.

Unlike a common coil which separates the conductors with an insulator in a rolled-up structure, the invention separates the wiring 200, 202 physically such that there is a space between the wiring 202 which can be surrounded by any gas or fluid within the item being sampled. More specifically, the flexible printed wiring board 100 has a first end 200 and a second end 202 extending from one opening 208 in the catheter 112. A connection 108 external to the catheter 112 joins the first end 200 to the second end 202 to form a loop 206. For example, the flexible circuitry 100 can be made from 2 mil kapton sheets with 1 mil laminated copper foil. In one example, the flexible printed wiring board 100 comprises a multilayer laminated structure that has four conductor (signal routing) layers. The top and bottom conductors are covered everywhere (except where solder pads 304 are located) with an insulator (e.g., kapton, etc.) layer to keep the conductors from shorting through the electrolyte medium (blood). The flexible printed wiring board 100 is connected to a transmission line 114 (single coaxial) by a connector 118. The transmission line 114 is connected to the receiver electronics (506 in FIG. 5). One ordinarily skilled in the art would understand that many other types of flexible printed wiring boards and printed circuit boards could be used, and that the invention is not limited to the examples discussed herein.

Independently moveable control rods 102 are connected to the first end 200 and the second end 202 to allow the first end 200 to be extended further out of the opening 208 than the second end 202 (to form the loop). More specifically, nonmagnetic steering wires 106 (e.g., stainless steel, titanium, etc.) extend out the other end of the catheter 112, and can be moved independently, thereby allowing independent movement of the control rods 102. The control rods 102 are preferably an electrical insulator to isolate the printed wiring board 100 from the conductive steering wire 106.

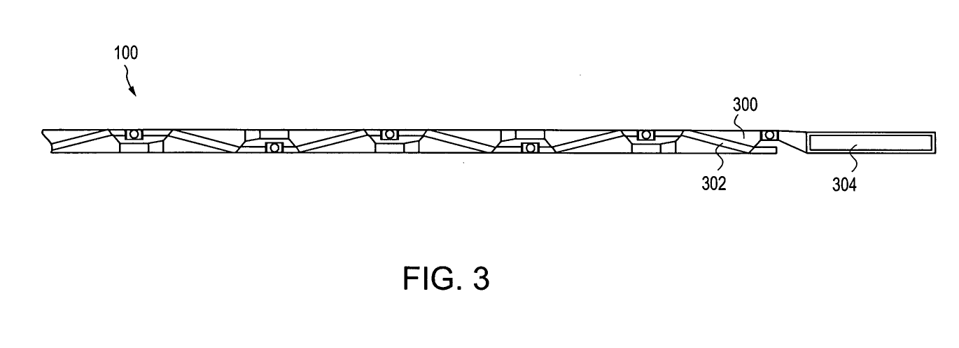

The flexible printed wiring board 100 includes capacitors 110 on the second end 202 of the flexible printed wiring board 100. The invention uses serial and parallel capacitors to match to the resistance/impedance of the receiver. In one example, the loop uses two serial capacitors and one parallel capacitor to provide a near 50 ohm match to the receiver. In the example shown in FIG. 3, the two series capacitors, used for matching the coil to a 50 ohm load (that of the receiver 506), are distributed around the loop of the coil in such a way as to improve the coil's Q while preserving the capacitance match. A parallel capacitor is used to complete the 50 ohm match to the receiver 506 input. The capacitors can be small chip capacitors with non-magnetic properties. By placing the capacitors 110 as close as possible to the coil area 206, the invention essentially eliminates any affect the receiver cable 114 will have on the coil. Therefore, because the invention places the capacitors 110 very close to the coil area 206, the radio frequency coil 206 is effectively isolated from outside sources of interference.

The flexible printed wiring board 100 can also include shielding circuitry 302 (e.g., a Faraday shield, etc.) on the flexible printed wiring board 100. The printed wiring board 100 comprises a flat ribbon shaped conductor (such as a strip of copper, etc. 300) that includes printed structures thereon (e.g., the shield 302, capacitors 110, etc.). FIG. 3 also illustrates the solder pad 304 that is useful in making the connection 108 to form the loop 206. The inventive shielded coil is useful for picking up magnetic lines of flux in a noisy electric field environment.

The Faraday shield 302 illustrated in FIG. 3 is incorporated into the printed wiring board 100 to help eliminate the background noise and distinguish it from the responsive emission signal (magnetic lines of flux). In one example, this shield comprises a Faraday-type shield 302 that is connected to ground and broken at one portion of the coil to allow flux lines to induce a current on the coil. As discussed in U.S. Pat. No. 5,699,801, incorporated herein by reference, a Faraday shield comprises a plurality of rings, and is used to limit interference. Thus, using the shield shown in FIG. 3, the inventive coil is protected from the electric field of eddy currents generated in the electrolyte medium. The shield should not be too capacitive or else the resonant frequency of the coil will be shifted too close to the resonant frequency of the MRI system (˜64 MHz) which would reduce Q of the coil (which reduces sensitivity of the image).

Therefore, the printed wiring board 100 can include one or more individual conductors (wires) and the resistance can be adjusted by including one or more serial and parallel capacitors 110 depending upon the nature of the coil required in light of the specific application. FIG. 3 illustrates a single coil conductor 300; however, one ordinarily skilled in the art will understand that a single flexible printed wiring board (or flexible printed circuit board (PCB)) that includes multiple conductors (or multiple layers of conductors) separated by insulators could be used in different applications of the invention.

The insulator on the outer layers of the printed wiring board 100 can be altered (made thicker or thinner) in different areas to change the flexibility of different portions of the printed wiring board 100 in order to urge the coil to deflect as desired for the application. Therefore, the upper portion of the printed wiring board 200 is substantially more flexible than the lower portion 202, which causes the upper portion 200 to form the loop 206 shown in FIG. 2. In this example, the upper portion 200 can be described as flexible or substantially flexible while the lower portion 202 can be described as non-flexible, rigid, stiff, etc. Therefore, while the printed wiring board 100 is a continuous flat belt-shaped (e.g., ribbon like) item that is soldered at connection 108 to itself in order to form a loop, different sections of the structure can have different properties (such as different coefficients of flexibility).

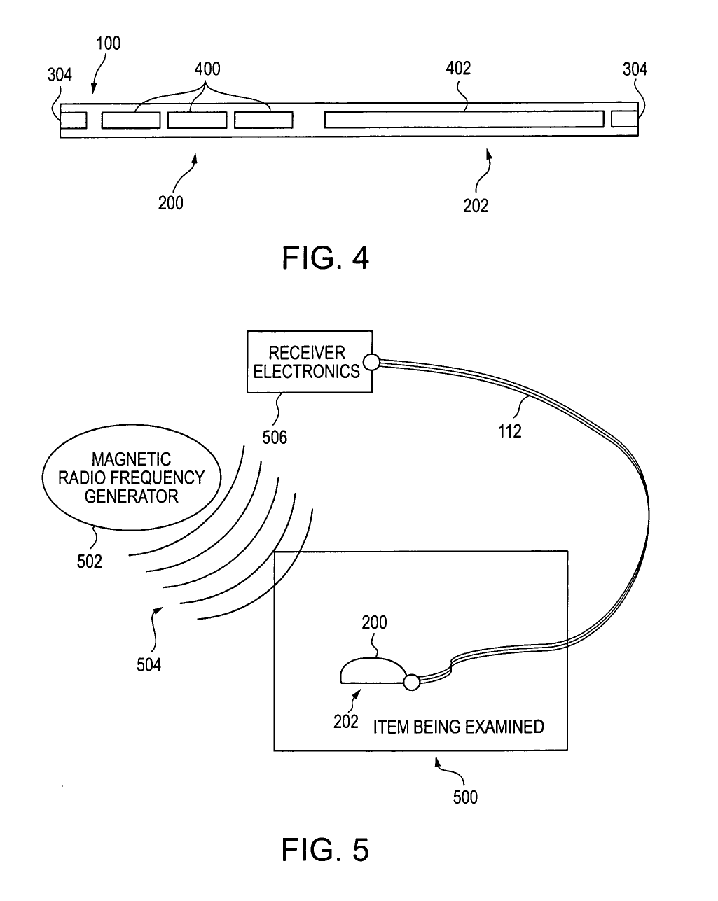

Thus, the first end 200 is more flexible than the second end 202, and the relative flexibility of the first end 200 with respect to the second end 202 causes the first end 200 to take the shape of a round arc when extended from the catheter 112. In addition, different insulator sections and thicknesses on the flexible printed wiring board 100 help define the shape of the loop. More specifically, as shown in the top view of the flexible printed wiring board 100 in FIG. 4, the more flexible first end 200 can include one or multiple insulator sections 400 formed on the flexible printed wiring board 100. To the contrary, a continuous insulator section 402 can be formed over the second end 202 to make the second end 202 less flexible than the first end. Additionally, the one or more insulator sections 400 formed on the more flexible first end 200 can be made thinner or made with less insulator layers when compared to the other insulator 402 so that the insulator sections 400 are more flexible than the insulator section 402.

As shown in FIG. 5, once the catheter 112 is inside or directly adjacent to (e.g., on or touching) the item 500 that is to be imaged (e.g., an organ, such as a heart, or other small, delicate, animate or inanimate object) the flexible wiring board 100 is moved through the catheter 112 by pushing both steering wires 106 relative to the catheter 112 until the flexible wiring board 100 partially extends out of the opening 208 of the catheter 112. By moving one steering wire 106 with respect to the other, one of the control rods 102 attached to the upper portion 200 of the printed wiring board 100 is extended further out of the opening 208 of the catheter 112. This difference in movement between the upper portion of the printed wiring board 200 and the lower portion of the printed wiring board 202 forms the loop or “coil area” 206 that acts as a radio frequency receiver coil for detecting the responsive emission signal (magnetic lines of flux) 504 from the magnetic radio frequency generator 502 to provide the imaging in the MRI device to the receiver electronics 506.

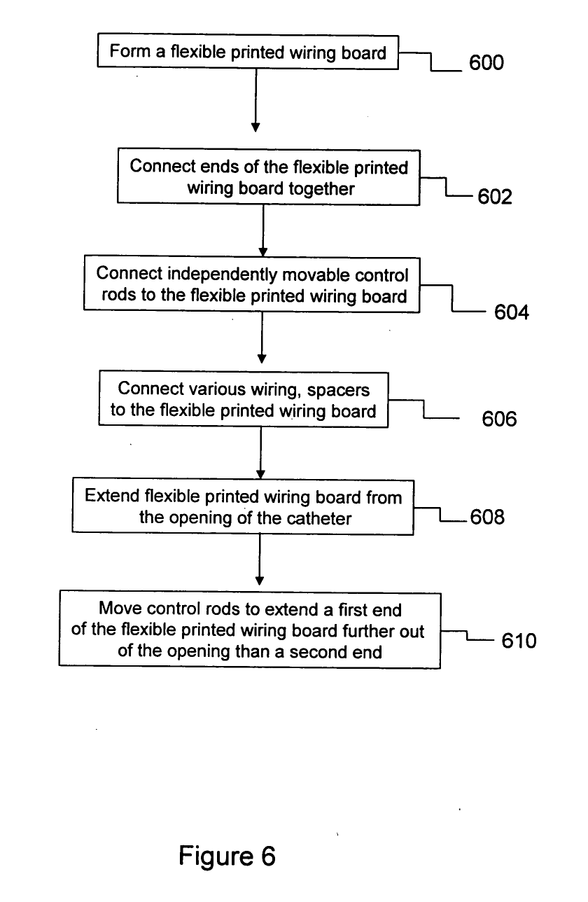

As shown in flowchart form in FIG. 6, the invention provides a method of manufacturing this radio frequency receiver coil. This method first forms a flexible printed wiring board 600 (that includes forming shielding circuitry on the flexible printed wiring board) and connects ends of the flexible printed wiring board together 602. The independently movable control rods are connected to the flexible printed wiring board next 604. Various wiring, spacers 104, etc. are then connected to the flexible printed wiring board (606) to complete the coil. The flexible printed wiring board is then positioned within a catheter (608) such that the ends of the flexible printed wiring board extend from the opening of the catheter. The control rods are moved to extend a first end of the flexible printed wiring board further out of the opening than a second end of the flexible printed wiring board (610) such that the portion of the flexible printed wiring board outside the opening forms a loop.

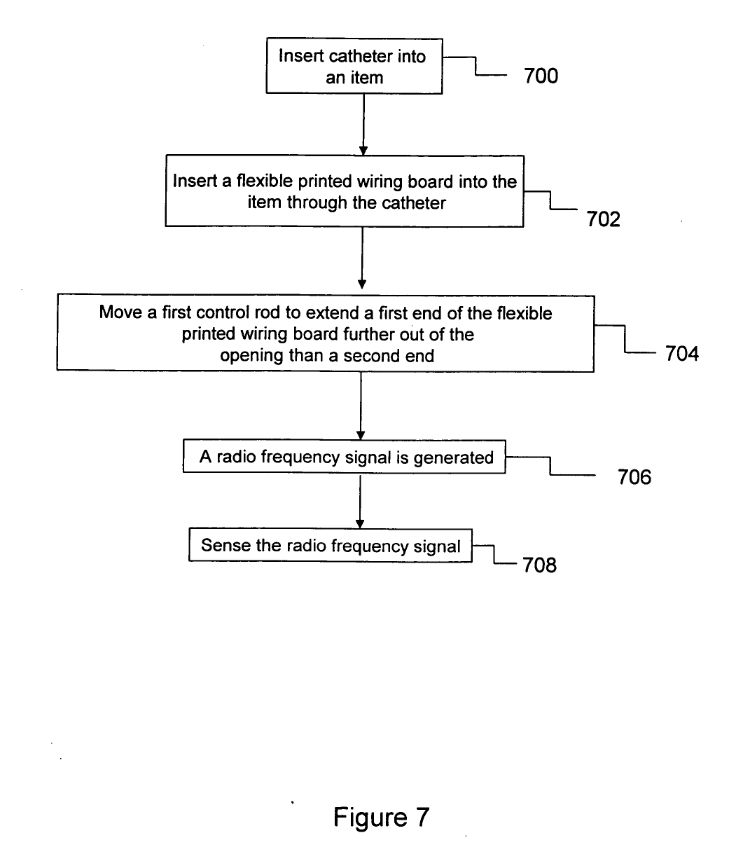

FIG. 7 illustrates the inventive method of performing magnetic resonance imaging (MRI) with the structure shown in FIGS. 1-5. In this methodology, a catheter is inserted into an item (700), such that an opening at one end of the catheter is positioned within the item. Next, the radio frequency coil comprising a flexible printed wiring board is inserted into the item through the catheter (702). A first control rod is moved to extend a first end of the flexible printed wiring board further out of the opening than a second end of the flexible printed wiring board (704), such that the portion of the flexible printed wiring board outside the opening forms a loop. Then, a radio frequency signal (706) is generated outside the item, and the invention senses the radio frequency signal (708) using the radio frequency coil.

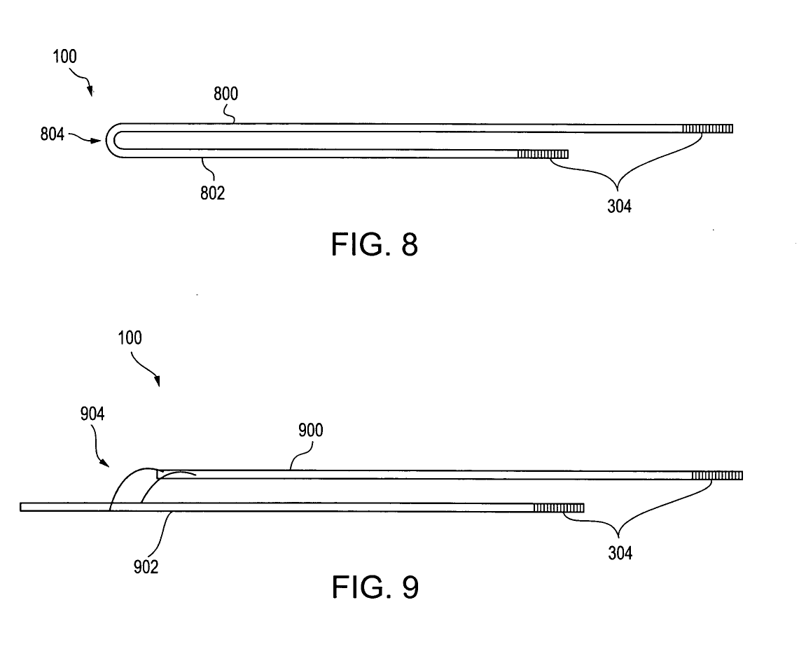

The flexible printed wiring board 100 can comprise a single piece (one piece) or a two piece design. For example, as shown in FIG. 8, the flexible printed wiring board 100 can comprise a single piece of material that includes a more flexible end 800 a second end 802 that is less flexible. These first and second ends 800, 802 correspond to the first and second ends 200, 202, discussed above. In this embodiment, the single piece flexible printed wiring board curves (bends) at region 804. To the contrary, the flexible printed wiring board 100 shown in FIG. 9 comprises a more flexible printed wiring board 900, and a less flexible printed wiring board 902 that are connected by wiring 904. These first and second boards 900, 902 also correspond to the first and second ends 200, 202, discussed above. The invention provides both the single piece and two piece designs so as to allow flexibility during the manufacturing process and provide the most efficient manufacturing system, depending upon the specific manufacturing environment.

Electrically, the flat conductor has advantages over a round wire design. More particularly, the flat shape provides an increase in conductor surface area, and an increase in ease of manufacturing associated with including a Faraday shield. Also, the printed circuit board nature of the process allows easy integration of component attachment and wire connections.

Loops used outside or away from the item being imaged cannot produce the resolution and quality of images available with the invention, because the inventive coil can be placed directly on or within the item being imaged. For example, a coil that is used outside a human organ (such as a heart) cannot provide the resolution and quality that is achievable with the invention in, for example, MRI-assisted cardiac procedures. Further, the deployable flat conductor coil design can be used to tailor the size of the loop to the specific application (e.g., different sized loops could be used when imaging an atrium verses a ventricle within a heart) by changing flexibility, flexible printed wiring board length, etc., as discussed above.

The invention has a unique low capacitance Faraday shield design which facilitates a thin coil. The coil is extremely narrow and can be inserted, for example, into an item as small as (or smaller than) an 8 French catheter. The flexible printed wiring board design is also inexpensive and easy to manufacture. The use of serial capacitors improves electrical performance. The capacitance matching of the coil to the MRI system additionally improves signal to noise ratio.

While the invention has been described in terms of preferred embodiments, those skilled in the art will recognize that the invention can be practiced with modification within the spirit and scope of the appended claims.

What is claimed is:

1. A radio frequency receiver coil adapted to be extended from a catheter, said coil comprising a flexible printed wiring board comprising:
a first end of said flexible printed wiring board extending from an opening in said catheter;
a second end of said flexible printed wiring board extending from said opening in said catheter; and
a connection external to said catheter joining said first end to said second end to form a loop.

2. The coil in claim 1, wherein said flexible printed wiring board has a flat ribbon shape.

3. The coil in claim 1, wherein said first end is more flexible than said second end.

4. The coil in claim 3, wherein the relative flexibility of said first end with respect to said second end causes said first end to take the shape of a round arc when extended from said catheter.

5. The coil in claim 1, further comprising control rods connected to said first end and said second end, wherein said control rods are independently moveable.

6. The coil in claim 1, wherein said flexible printed wiring board includes capacitors adjacent said second end.

7. The coil in claim 1, further comprising insulator sections on said flexible printed wiring board, wherein said insulator sections define the shape of said loop.

8. A radio frequency receiver coil adapted to be extended from a catheter, said coil comprising a flexible printed wiring board comprising:
a first end of said flexible printed wiring board extending from an opening in said catheter;
a second end of said flexible printed wiring board extending from said opening in said catheter;
a connection external to said catheter joining said first end to said second end to form a loop; and
shielding circuitry on said flexible printed wiring board.

9. The coil in claim 8, wherein said shielding circuitry comprises a Faraday shield.

10. The coil in claim 8, wherein said first end is more flexible than said second end.

11. The coil in claim 10, wherein the relative flexibility of said first end with respect to said second end causes said first end to take the shape of a round arc when extended from said catheter.

12. The coil in claim 8, further comprising control rods connected to said first end and said second end, wherein said control rods are independently moveable.

13. The coil in claim 8, wherein said flexible printed wiring board includes capacitors adjacent said second end.

14. The coil in claim 8, further comprising insulator sections on said flexible printed wiring board, wherein said insulator sections define the shape of said loop.

15. A method of manufacturing a radio frequency receiver coil, said method comprising:
forming a flexible printed wiring board;
connecting ends of said flexible printed wiring board together;
connecting control rods to said flexible printed wiring board, wherein said control rods are independently moveable;
positioning said flexible printed wiring board within a catheter such that the ends of said flexible printed wiring board extend from the opening of said catheter; and
moving said control rods to extend a first end of said flexible printed wiring board further out of said opening than a second end of said flexible printed wiring board such that the portion of said flexible printed wiring board outside said opening forms a loop.

16. The method in claim 15, wherein said flexible printed wiring board has a flat ribbon shape.

17. The method in claim 15, wherein said first end is more flexible than said second end.

18. The method in claim 17, wherein the relative flexibility of said first end with respect to said second end causes said first end to take the shape of a round arc when said first end is extended further out of said opening than said second end.

19. The method in claim 15, wherein said process of forming said flexible printed wiring board includes forming capacitors adjacent said second end.

20. The method in claim 15, further comprising forming insulator sections on said flexible printed wiring board, wherein said insulator sections define the shape of said loop.

21. A method of manufacturing a radio frequency receiver coil, said method comprising:
forming a flexible printed wiring board;
forming shielding circuitry on said flexible printed wiring board;
connecting ends of said flexible printed wiring board together;
connecting control rods to said flexible printed wiring board, wherein said control rods are independently moveable;
positioning said flexible printed wiring board within a catheter such that the ends of said flexible printed wiring board extend from the opening of said catheter; and
moving said control rods to extend a first end of said flexible printed wiring board further out of said opening than a second end of said flexible printed wiring board such that the portion of said flexible printed wiring board outside said opening forms a loop.

22. The method in claim 21, wherein said shielding circuitry comprises a Faraday shield.

23. The method in claim 21, wherein said flexible printed wiring board has a flat ribbon shape.

24. The method in claim 21, wherein said first end is more flexible than said second end.

25. The method in claim 24, wherein the relative flexibility of said first end with respect to said second end causes said first end to take the shape of a round arc when said first end is extended further out of said opening than said second end.

26. The method in claim 21, wherein said process of forming said flexible printed wiring board includes forming capacitors adjacent said second end.

27. The method in claim 21, further comprising forming insulator sections on said flexible printed wiring board, wherein said insulator sections define the shape of said loop.

28. A catheter comprising:
an enclosed section having an opening;
a radio frequency receiver coil adapted to be extended from said opening of said catheter, said coil comprising a flexible printed wiring board comprising:
a first end of said flexible printed wiring board extending from an opening in said catheter;
a second end of said flexible printed wiring board extending from said opening in said catheter; and
a connection external to said catheter joining said first end to said second end to form a loop.

29. The catheter in claim 28, wherein said flexible printed wiring board has a flat ribbon shape.

30. The catheter in claim 28, wherein said first end is more flexible than said second end.

31. The catheter in claim 30, wherein the relative flexibility of said first end with respect to said second end causes said first end to take the shape of a round arc when extended from said catheter.

32. The catheter in claim 28, further comprising control rods connected to said first end and said second end, wherein said control rods are independently moveable.

33. The catheter in claim 28, wherein said flexible printed wiring board includes capacitors adjacent said second end.

34. The catheter in claim 28, further comprising insulator sections on said flexible printed wiring board, wherein said insulator sections define the shape of said loop.

35. A catheter comprising:
an enclosed section having an opening;
a radio frequency receiver coil adapted to be extended from said opening of said catheter, said coil comprising a flexible printed wiring board comprising:
a first end of said flexible printed wiring board extending from an opening in said catheter;
a second end of said flexible printed wiring board extending from said opening in said catheter;
a connection external to said catheter joining said first end to said second end to form a loop; and
shielding circuitry on said flexible printed wiring board.

36. The catheter in claim 35, wherein said shielding circuitry comprises a Faraday shield.

37. The catheter in claim 35, wherein said first end is more flexible than said second end.

38. The catheter in claim 37, wherein the relative flexibility of said first end with respect to said second end causes said first end to take the shape of a round arc when extended from said catheter.

39. The catheter in claim 35, further comprising control rods connected to said first end and said second end, wherein said control rods are independently moveable.

40. The catheter in claim 35, wherein said flexible printed wiring board includes capacitors adjacent said second end.

41. The catheter in claim 35, further comprising insulator sections on said flexible printed wiring board, wherein said insulator sections define the shape of said loop.

42. A method of performing magnetic resonance imaging (MRI), said method comprising:
inserting a catheter into an item, such that an opening at one end of said catheter is positioned within said item;
inserting a radio frequency coil comprising a flexible printed wiring board into said item through said catheter;
moving a first control rod to extend a first end of said flexible printed wiring board further out of said opening than a second end of said flexible printed wiring board, such that the portion of said flexible printed wiring board outside said opening forms a loop;
generating a radio frequency signal outside said item; and
sensing said radio frequency signal using said radio frequency coil.

43. The method in claim 42, wherein said flexible printed wiring board has a flat ribbon shape.

44. The method in claim 42, wherein said first end is more flexible than said second end.

45. The method in claim 44, wherein the relative flexibility of said first end with respect to said second end causes said first end to take the shape of a round arc when said first end is extended further out of said opening than said second end.

46. The method in claim 42, wherein said flexible printed wiring board includes capacitors adjacent said second end.

47. The method in claim 42, wherein insulator sections on said flexible printed wiring board define the shape of said loop.
